(19) United States
(12) Patent Application Publication
(10) Pub. No.: US 2017/0226077 A1
(43) Pub. Date: Aug. 10, 2017

(54) Production of 11-Hydroxy-THC

(71) Applicant: Robin James Bach, Tucson, AZ (US)

(72) Inventor: Robin James Bach, Tucson, AZ (US)

(21) Appl. No.: 14/998,738

(22) Filed: Feb. 9, 2016

(51) Int. Cl. C07D 311/80

(52) U.S. Cl. CPC C07D 311/80

(57) ABSTRACT

The invention involves a method of producing 11-Hydroxy-THC by first creating a mixture of oil and cannabis material. This mixture is subjected to a simultaneous process of high pressurization and heat. Ideally the cannabis chosen is from a kief created by freezing a cannabis plant part and vibrating/screening the frozen cannabis plant part.

### BACKGROUND OF THE INVENTION

This invention relates to the extraction of enhanced chemicals from cannabinoids and more particularly to the production of 11-Hydroxy-THC.

11-Hydroxyl-THC-delta9-tetrahydrocannabinol (11-OH-THC) is the main active metabolite of THC which is formed in the body after cannabis consumption. 11-OH-THC is active in its own right with the affects not necessarily identical to those of THC. 11-OH-THC is more potent than THC and crosses the blood-brain barrier more easily.

Interestingly, peak THC concentrations are lower after oral administration rather than smoked administration, while 11-OH-THC ratios are reversed, higher after oral over smoked cannabis.

11-OH-THC is subsequently metabolized further to 11-nor-9-carboxy-THC which is not psychoactive and is thought to play a role in the analgesic and anti-inflammatory effects of cannabis.

Because of this, there are many medical applications which are more suited for 11-OH-THC which means that its refinement and purification is sought.

Unfortunately, this refinement is not always easily or efficiently done which means that the consumer costs for 11-nor-9-carboxy-THC is often prohibitive. Many medical applications for 11-nor-9-carboxy-THC are then not considered or attempted.

It is clear from the foregoing that there is a need to efficiently extract 11-Hydroxy-THC.

### SUMMARY OF THE INVENTION

The invention involves a method of producing 11-Hydroxy-THC by first creating a mixture of oil and cannabis material. This mixture is subjected to a simultaneous process of high pressurization and heat. Ideally the cannabis chosen is from a kief created by freezing a cannabis plant part and, vibrating screening frozen cannabis plant part.

Within this discussion, tetrahydrocannabinol (THC), a main isomer of delta-9-tetrahydrocannabinol, and is a source for the production of 11-Hyrodxy-THC.

More specifically, the invention provides for a method for producing 11-Hydroxy-THC. The process/method first creates a mixture of oil and cannabis material. This mixture is subjected both high pressure and high heat for a period between 30 and 50 minutes. In the preferred embodiment, the pressure applied is up to 750 pounds per square inch; and, is heated to a minimum of 240 degrees Fahrenheit.

In some application, the cannabis material is first formed into a kief. Kief (sometimes referred to as “keef” or Kif”) is the resin glands or trichomes of cannabis which are often obtained from dried cannabis flowers which are ground and passed through a mesh screen or sieve.

In the present invention, the preferred method for producing the kief is to first freeze the cannabis plant part, and then pass the frozen cannabis plant part through a vibrating screen to obtain the kief

In another embodiment, the cannabis material is a hashish. Hashish is also produced from the resin gland (trichomes) and is typically solid or resinous.

To heighten the amount of 11-Hydroxy-THC produced, in the preferred embodiment, the source cannabis is chosen to have an enhanced THC content. The source cannabis is also ideally washed at least twice with methanol. This washed cannabis is further refined before processing by a cooking process to obtain a consistency which has the optimal desired result when subjected to the pressurization/heating process described above.

In some embodiments, the source cannabis material chosen has an enhanced cannabidiol (CBD). CBD is one of several active cannabinoids which has been associated with cannabis.

Cannabidiol (CBD) is an active cannabinoid identified in cannabis and is considered to have a wider scope of medical applications than THC. Traditionally, CBD is administered in a liquid form for treatment of Dravet syndrome, a form of epilepsy that are often triggered by hot temperatures or fever within the first year of a child's life.

In this embodiment, the CBD process, the source cannabis is washed, at least twice with ethanol and then purged with water. This washed cannabis is then cooked to a desired consistency which will enhance the subsequent pressurization/heating of the material.

The invention, together with various embodiments will be more fully explained by the accompanying drawings and the following description thereof.

### DRAWINGS IN BRIEF

FIGS. 1A and 1B illustrate the preferred process for the production of 11-Hydroxy-THC.

FIGS. 2A and 2B illustrate the production of kief used in an alternative embodiment for the production of 11-Hydroxy-THC.

FIG. 3 illustrates the washing of cannabis as a preparation step for the preferred embodiment.

### DRAWINGS IN DETAIL

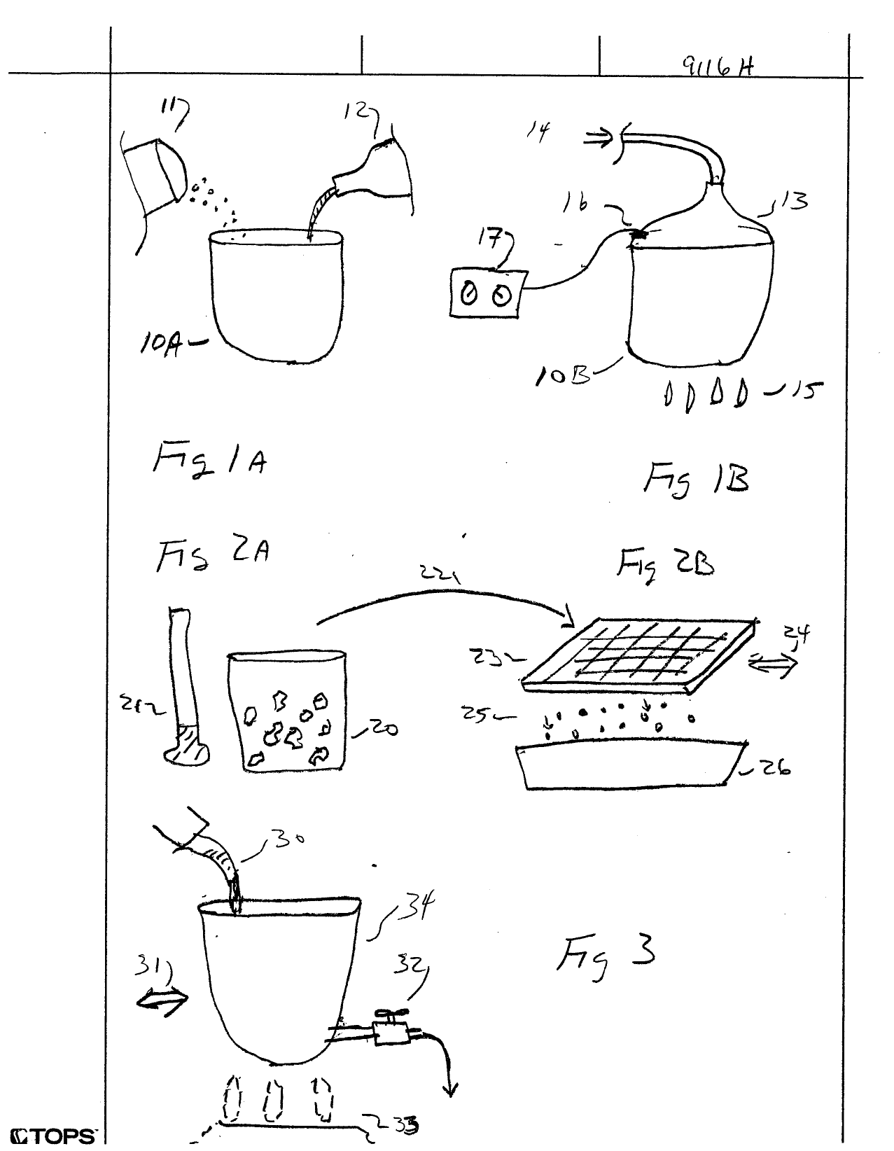

FIGS. 1A and 1B illustrate the preferred process for the production of 11-Hydroxy-THC.

As illustrated in FIG. 1A, cannabis 11 and oil 12 are combined in a vessel 10A. The mixing process is not illustrated but can be done mechanically with a mixing wand or by agitating vessel 10A.

As shown in FIG. 1B, vessel 10B is sealed using lid 13. This forms an air tight container into which pressure 14 is applied and heat 15. The mixture experiences pressurization of up to 750 pound per square inch and a heating of a minimum of 240 degrees Fahrenheit. The pressurization and heating as outlined is to continue for a period of 30-50 minutes.

The resulting material has a higher concentration of 11-Hydroxy THC.

In some embodiments, instead of raw cannabis 11, the cannabis is first rendered into hashish. Those of ordinary skill in the art recognize how hashish is made.

Additionally, in some embodiments, the raw cannabis is chosen for its inherit content of either THC or CBD depending on the overall result sought.

FIGS. 2A and 2B illustrate the production of kief used in an alternative embodiment for the production of 11-Hydroxy-THC. Kief is used in lieu of the raw cannabis outlined relative to FIGS. 1A and 1B.

As illustrated in FIG. 2A, the raw cannabis 20 is first frozen 21. The frozen cannabis is transferred 22 to screen 23 which is vibrated 24 to break up the frozen cannabis and to classify the material 25 into sought after size ranges for collection 26.

FIG. 3 illustrates the washing of cannabis as a preparation step for the preferred embodiment.

In some embodiments of the invention, instead of raw cannabis, the material is first prepared by two washings using a selected liquid 30 which is passed through the raw cannabis (not shown) within container 34. The chosen liquid is agitated 31 within container 34 before being discharged from container 34 via valve 32.

In some embodiments of this process, the washed material is heated 33 to obtain the desired consistency.

If the chosen cannabis is high in CBD, the preferred liquid chosen 30 for the washing is ethanol. This washing is done twice and then the cannabis is rinsed with water and then heated/dried to the desired consistency. This ideally involves boiling the cannabis/water mixture.

If the chosen cannabis is high in THC, the preferred liquid chosen 30 is ideally methanol.

It is clear that the present invention provides an efficient mechanism for the extraction of 11-Hydroxy-THC.

What is claimed is:

1. A method of producing 11-Hydroxy-THC comprising the steps of:
a) Creating a mixture of oil and cannabis material; and,
b) for a period between 30 and 50 minutes, simultaneously,
1) subjecting the mixture to a pressure up to 750 pounds per square inch, and,
2) heating the pressurized mixture to a minimum of 240 degrees Fahrenheit, thereby obtaining 11-Hydroxy-THC.

2. The method of producing 11-Hydroxy-THC according to claim 1, wherein the step of creating the oil and cannabis material includes the step of creating kief from a cannabis plant.

3. The method of producing 11-Hydroxy-THC according to claim 2, wherein the step of creating kief includes the steps of:
a) freezing a cannabis plant part; and,
b) vibrating and screening the frozen cannabis plant part.

4. The method of producing 11-Hydroxy-THC according to claim 1, further including the step of creating the cannabis material by creating hashish.

5. The method of producing 11-Hydroxy-THC according to claim 1, further including the step of choosing a cannabis material having enhanced THC content prior to the step of creating a mixture of oil and cannabis material for the step of creating a mixture of oil and cannabis material.

6. The method of producing 11-Hydroxy-THC according to claim 5, further including the step of creating the cannabis material by the step of washing a source of cannabis at least twice with methanol to create the cannabis material.

7. The method of producing 11-Hydroxy-THC according to claim 6, further including the step of cooking a resultant product to a firmer state after the step of simultaneously subjecting the mixture to pressure and heating the pressurized material.

8. The method of producing 11-Hydroxy-THC according to claim 1, further including the step of choosing a cannabis material having enhanced CBD content prior to the step of creating a mixture of oil and cannabis.

9. The method of producing 11-Hydroxy-THC according to claim 8, further including the steps of creating the cannabis material by:
a) washing a source cannabis, at least twice, with ethanol; and,
b) purging the washed source cannabis with water to create the cannabis material.

10. The method of producing 11-Hydroxy-THC according to claim 9, further including a final step of cooking a resultant product to a desired consistency after the step of simultaneously subjecting the mixture to pressure and heating the pressurized material.

11. A method of producing 11-Hydroxy-THC comprising the steps of:
a) washing cannabis material with methanol;
b) boiling the mixture of cannabis material and methanol to remove the methanol;
c) purging a resulting mixture of cannabis material with,
1) ethanol, then
2) water, and then
3)
d) drying a purged resulting mixture of cannabis material to a desired consistency.

12. The method of producing 11-Hydroxy-THC according to claim 11, wherein the step of washing cannabis material with methanol includes the steps of draining methanol from the cannabis material and performing a second washing of the cannabis material with methanol.

13. A method of producing 11-Hydroxy-THC comprising the steps of simultaneously, for a period of between 30 and 50 minutes:
a) subjecting a mixture of oil and cannabis source to a pressure up to 750 pounds per square inch; and,
b) heating the pressurized mixture to a minimum of 240 degrees Fahrenheit.

14. The method of producing 11-Hydroxy-THC according to claim 13, further including the step of selecting the cannabis source from the group consisting of kief, hashish, plant material high in THC.

15. The method of producing 11-Hydroxy-THC according to claim 14, further including a step of cooking a resulting product to a desired consistency after the step of simultaneously subjecting the mixture to pressure and heating the pressurized material.

16. A method of producing 11-Hydroxy-THC comprising the steps of:
a) creating a prepared cannabis material by,
1) washing a cannabis material at least twice with methanol, and,
2) purging the cannabis with water which is dried to a desired consistency;
b) creating mixture of oil and prepared cannabis; and,
c) for a period between 30 and 50 minutes, simultaneously, subjecting the mixture to a pressure up to 750 pounds per square inch, and, heating the pressurized mixture to a minimum of 240 degrees Fahrenheit.

17. The method of producing 11-Hydroxy-THC according to claim 16, further including a final step of cooking the product to a desired consistency after the step of simultaneously subjecting the mixture to pressure and heating the pressurized material.
